(19) United States
(12) Patent Application Publication
(10) Pub. No.: US 2021/0145338 A1
(43) Pub. Date: May 20, 2021

(54) IOT BASED WEARABLE DEVICE, SYSTEM AND METHOD FOR THE MEASUREMENT OF MEDITATION AND MINDFULNESS

(71) Applicant: Rajlakshmi Borthakur, Bangalore (IN)

(72) Inventor: Rajlakshmi Borthakur, Bangalore (IN)

(21) Appl. No.: 16/630,451

(22) PCT Filed: Jul. 12, 2018
(86) PCT No.: PCT/IB2018/005515

§ 371 Date: Jan. 12, 2020

(30) Foreign Application Priority Data
Jul. 12, 2017 (IN) 201741001241

(51) Int. Cl. A61B 5/16; A61B 5/00; A61B 5/0205; G16H 20/70; G16H 40/67; G16H 50/30

(52) U.S. Cl. CPC A61B 5/165; A61B 5/7465; A61B 5/02055; A61B 5/0006; A61B 5/0008; A61B 2562/0271; G16H 20/70; G16H 40/67; G16H 50/30; A61B 2562/0219; A61B 5/7267

(57) ABSTRACT

An Internet of Things (IOT) system for management of a stress level and mental health of a human body. The system has one or more body sensors and a primary processing unit that runs an artificial intelligence system. The body sensors are adapted to measure at least one of a physiological parameter of the human body, body movement of the human body, or heat expenditure of the human body or combination thereof, and to generate a body data periodically or in realtime. The primary processing unit is adapted to receive and process the body data and adapted to determine at least one of the mental health of the human body and the stress level of the human body. The primary processing unit is adapted to provide therapies and give insights about the effectiveness of psychological therapies including CBD, meditation and mindfulness in a quantitative manner.

### FIELD OF THE INVENTION

The invention relates to the monitoring the state of health of a human body. More specifically, it relates to monitoring the physiological and psychological health of a human body based on the body data.

### BACKGROUND OF THE INVENTION

Most people these days lead stressed lives leading to various health conditions. Sometimes outcomes of stress are seen as lifestyle disorders like hypertension, diabetes, obesity and so on. Sometimes outcomes are manifested as mental health conditions, while for some individuals health-related changes are too subtle to detect. An effective way to reduce both physical and mental stress is by mental relaxation using various mental health therapies like psychotherapy, Cognitive behaviour therapies (CBTs), positive psychology-based therapies, meditation and mindfulness, and other psychologist-approved techniques.

Psychotherapy involves a variety of treatment options. During psychotherapy, a person with a mental illness talks to a trained mental health professional who helps him or her identify and work through the factors that may be triggering the illness. Cognitive behavioural therapy (CBT) is a short-term, goal-oriented psychotherapy treatment. The goal of CBT is to change patterns of thinking or behaviour in people to change the way they feel and react to situations. Positive psychology is “the scientific study of what makes life most worth living”, or “the scientific study of positive human functioning and flourishing on multiple levels that include the biological, personal, relational, institutional, cultural, and global dimensions of life”. Positive psychologists propose different ways in which happiness can be achieved. Social ties and networking, physical exercises and the practice of calming techniques like meditation contribute to happiness.

Through regular and supervised use of these techniques, a person could attain deep relaxation mentally and physically. Such psychology-based scientific techniques help in combating mental health conditions like chronic depression, severe anxiety, and various sleep disorders associated with stress. They also help people reduce the effect of lifestyle disorders. Psychologists generally use a “paper & pen” method to assess mental health issues and understand effectiveness of therapy. Further, psychologists depend on a person's narrative and own account to make sense of the extent of mental illness via subjective assessments. Questionnaires are traditional and albeit great tools used by psychologists to understand how a person deals with different emotions like happiness, sadness, hope, gratitude, satisfaction and so on. Mindful Attention Awareness Scale developed by Kirk Warren Brown, Ph.D. & Richard M. Ryan, Ph.D., Adult Hope Scale developed by C. R. Snyder, University Of Kansas, The Gratitude Questionnaire developed by Michael E. Mccullough, Ph.D., Robert A. Emmons, Ph.D., Jo-Ann Tsang, Ph.D. are some of the tools that can be cited in this respect. On the other hand for clinical assessments, tools like Generalized Anxiety Disorder-7 (GAD 7) is used for anxiety assessment, Patient Health Questionnaire-9 (PHQ 9) is used for depression, Trauma Screening Questionnaire (TSQ) and so on are used.

But while questionnaires are recommended by mental healthcare practitioners and are quite popularly used, there are no wearable or small form factor diagnostic and quantitative ways to measure emotions, mental stress or actual feelings of a person when he or she responds to such questionnaires. Similarly there are no integrated ways to connect physical and mental health diagnosis with effectiveness of therapies, treatment and medication in a scientific way using technology. Unlike the existence of innumerable tests for the diagnosis and determination of physical health condition, the absence of a quantitative way to diagnosis, prevent and manage mental health issues is a major challenge to the successful implementation of mental health programs. This inability to “see” changes is also one of the most common reasons why people give up their mental health practices and therapies, thereby deteriorating their existing condition.

U.S. application Ser. No. 13/154,022, discloses a brainwave actuated apparatus which captures brainwaves signal using a brainwave sensor and further determine characteristics of the brainwave signal. It has limitations with respect to accuracy of captured brainwave, and cannot properly identify quality of meditation.

U.S. application Ser. No. 11/657,831, discloses a wearable relaxation inducing apparatus which includes either a harness or a garment made of elastically flexible fabric tightly worn on the torso, electromechanical sensors attached to the fabric translating the breathing movements of a wearer into electric signals representing breathing rate and depth, and electrically operated transducers attached to the fabric providing tactile feedback to the body about breathing. Breathing analysis gives limited information about the quality of meditation, and leaves out certain vital information for analysis of meditation quality.

U.S. application Ser. No. 15/043,330, discloses method and apparatus for providing biofeedback during a meditation exercise. The wearable device includes one or more biometric sensors and a user interface. The method involves prompting the user, via the user interface, to perform a meditation exercise, the meditation exercise being associated with a target physiological metric related to the physiology of the user. The method further involves measuring, based on output of at least one of the one or more biometric sensors, a physiological metric of the user during the meditation exercise. The method further involves determining a performance score indicating the user's performance during the meditation exercise based on comparing the measured physiological metric with the target physiological metric. The method further involve providing, via the user interface, based on the performance score, feedback information indicative of the user's performance during the meditation exercise. However, the method and apparatus do not captures important parameters like body temperature, and other such parameters captured from skin surface, and further the physiological metric generated is inaccurate due to collection location of biometric sensors, and the way metric are generated. Hence, the score calculated is not optimal.

The technological solutions available in the market today to test and manage mental health issues use very few parameters to give inputs regarding the physiological state of users. None of them give direct indication of the overall health and well-being of a person by integrating elements of mental and physical health. Nor do they take into account known stressors and reactions, various psychological triggers, environmental and geographical impact on the user or the impact of different types of medication on changing behaviour. The present invention addresses the aforementioned problems by means of a system which is capable of managing the physical and mental health of an individual.

### OBJECT OF THE INVENTION

It is an object of the invention to provide a technique for managing the stress level and mental health of a user by determining quantitatively the effects of various factors on the physiological and psychological health of the user.

### SUMMARY OF THE INVENTION

The object of the invention is achieved by a system for management of a stress level and mental health of a human body. The system has one or more body sensors and a primary processing unit. The body sensors are adapted to measure at least one of a physiological parameter of the human body, body movement of the human body, or heat expenditure of the human body or combination thereof, and to generate a body data. The primary processing unit is adapted to receive and process the body data and adapted to determine at least one of the mental health of the human body and the stress level of the human body.

According to one embodiment of the system, the primary processing unit is adapted to process the body data by comparing the body data with one or more reference values, and adapted to determine at least one of the mental health of the human body and the stress level of the human body

According to another embodiment of the system, the primary processing unit is adapted to process the body data and to generate a body score related to at least one of overall health, mental health, physical health, heart health, sleep, or human body activity, or combination thereof.

According to yet another embodiment of the system, one or more physiological parameter of the human body is measured using at least one of electrodermal activity sensor, skin temperature sensor, photoelectric plethysmography sensor, electro cardiogram sensor, or electromyography sensor, or combination thereof.

According to a further embodiment of the system, the body movement of the human body is measured by a 9-axis motion sensor which comprises a 3-axis gyroscope, 3-axis accelerometer and 3-axis magnetometer.

According to one embodiment of the system, the system has a data extraction unit functionally coupled to the one or more body sensors, and to extract the body data from the body sensors.

According to another embodiment of the system, the system has a database adapted to receive and store at least one of the body data, the body score, or the determination of at least one of the mental health of the human body and the stress level of the human body.

According to yet another embodiment of the system, the system has an IoT unit adapted to establish communication to the database, which is placed remotely to the IoT module, and adapted to send at least one of the body data, the body score, or the determination of at least one of the mental health of the human body and the stress level of the human body to the database.

According to a further embodiment of the system, the system has an input unit adapted to receive inputs related to validation of at least one of the body data, the body score, or the determination of at least one of the mental health of the human body and the stress level of the human body.

According to one embodiment of the system, the system has one or more environmental sensors adapted to generate a contextual data related to at least one of interactions of the human body with other human bodies, geographic location visited by the human body, environment of the human body, or food and nutrition habits of the human body, or combination thereof. The primary processing unit is adapted to process the body data along with the contextual data for at least one of the determination of at least one of the mental health of the human body and the stress level of the human body, and generation of the body score.

According to another embodiment of the system, the system has a Machine Learning and Artificial Intelligence processing unit adapted to receive and process the body data and the contextual data, adapted to identify one or more patterns of the human body based on processing of the body data and the contextual data and adapted to compare the patterns of the human body, and to determine risks related to stress level and mental health of a human body.

According to yet another embodiment of the system, the Machine Learning and Artificial Intelligence processing unit is adapted to receive various stimulus and/or therapies administered to the human body, and to generate responses to each of the stimulus and/or therapies by the human body based on the body data.

According to a further embodiment of the system, the Machine Learning and Artificial Intelligence processing unit is adapted to process the responses to each of the stimulus and/or therapies by the human body and adapted to generate risk stratification of the human body.

According to another embodiment of the system the Machine Learning and Artificial Intelligence processing unit is adapted to generate therapeutic recommendation based on at least processing of the responses to the stimulus and/or therapies by the human body based on the body data, body data, or the contextual information, or combination thereof.

According to one embodiment of the system, the system has a device which encapsulates at least one of the sensor/s, the primary processing module, the data extraction unit, the database, IoT unit, the input unit, or, the Machine Learning and Artificial Intelligence processing unit, or the combination thereof, wherein the device is wearable on at least one of the body parts.

According to another embodiment of the system, the device is wearable on hand or feet of the human body.

According to yet another embodiment of the system, the device further has a rechargeable battery and a battery management unit, the battery management unit is adapted to at least monitor a physical state and/or chemical state of the battery, to control operating environment of the battery, or combination thereof.

According to another embodiment of the system, the device further comprises an output unit adapted to render at least one of the body data, the body score, or the determination of at least one of the mental health of the human body and the stress level of the human body.

According to a further embodiment of the system, the device processing unit is adapted to be remotely coupled for communication to a telemedicine platform for enabling data flow between the device and the telemedicine platform.

### BRIEF DESCRIPTION OF DRAWINGS

FIG. 1 illustrates a system for managing the stress level and mental health of a user

FIG. 2 illustrates a flow diagram for determining the stress level and mental health of a user

FIG. 3 illustrates the wearable sensors

### DETAILED DESCRIPTION

The best and other modes for carrying out the present invention are presented in terms of the embodiments, herein depicted in Drawings provided. The embodiments are described herein for illustrative purposes and are subject to many variations. It is understood that various omissions and substitutions of equivalents are contemplated as circumstances may suggest or render expedient, but are intended to cover the application or implementation without departing from the spirit or scope of the present invention. Further, it is to be understood that the phraseology and terminology employed herein are for the purpose of the description and should not be regarded as limiting. Any heading utilized within this description is for convenience only and has no legal or limiting effect.

The terms “a” and “an” herein do not denote a limitation of quantity, but rather denote the presence of at least one of the referenced item.

It will be understood by those skilled in the art that the foregoing general description and the following detailed description are exemplary and explanatory of the invention and are not intended to be restrictive thereof.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more sub-systems or elements or structures or components preceded by “comprises . . . a” does not, without more constraints, preclude the existence of other, sub-systems, elements, structures, components, additional sub-systems, additional elements, additional structures or additional components. Appearances of the phrase “in an embodiment”, “in another embodiment” and similar language throughout this specification may, but not necessarily do, all refer to the same embodiment.

The present invention focuses on a mechanism for determining the mental health and stress levels of a user and also manages the mental health and stress issues of a user. Although, at present there are multiple remedies available that pertain to detection of the state of mental health, few of them take into consideration the multitude of factors which could lead to increase in stress levels for an individual and thereby a deterioration in mental health. Further, each factor affects different individuals in different ways therefore a standard remedy cannot suffice for everyone. The present invention addresses these concerns by taking physiological and environmental factors into consideration for determining the mental health of a user.

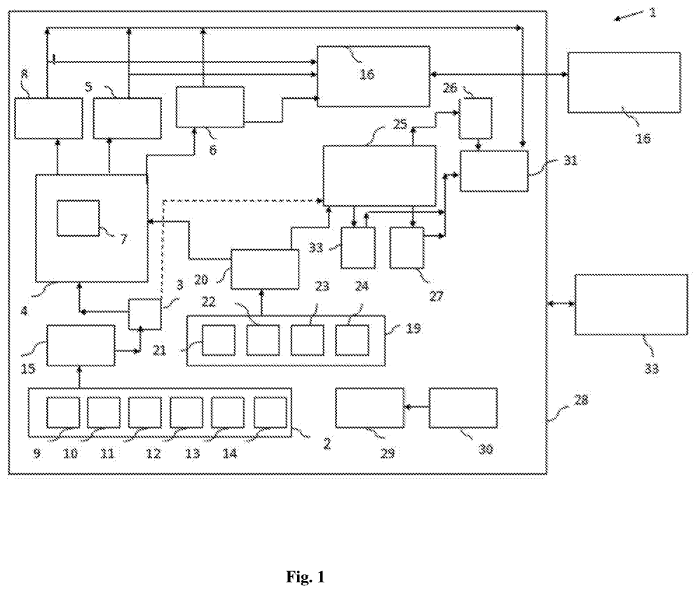

In one implementation of the invention, technique is provided for managing the mental health and stress levels of a user. Such an implementation is shown in FIG. 1.

FIG. 1 illustrates a system 1 for determining and managing the stress level and mental health of a user. The system has sensors 3, 19 and a primary processing unit 4. The provided sensors 3, 19 are of two types—body sensors 2 which detect the physiological parameters of the human body and environmental sensors 19 which detect the elements in whose vicinity the human body is present. The body sensors include Electrodermal activity (EDA) sensor 9, skin temperature sensor 10, photoelectric plethysmography sensor (PPG) 11, electro cardiogram sensor (ECG) 12, 9-axis motion sensor 14 and electro myography sensor (EMG) 13. EDA sensor 9 facilitates in the measurement of the sweat gland activity, through which the emotional arousal of a human body can be detected as the EDA 9 is activated automatically by the sweat glands in the skin. PPG 101 is an optical measurement technique for measuring the heart rate, it also measures the SPO2 level, respiration rate, Heart Rate Variability (HRV) and the Blood Pressure (BP) of a person. ECG 12 measures the measures the electrical activity of the heart while EMG 13 monitors the electric signal from muscles, which is controlled by the nervous system and produced during muscle contraction. In addition to this 9-axis motion sensor 14 tracks the movements of an individual accurately while the skin temperature 10 provides the temperature of an individual consistently. The aforementioned data is involuntary which a human body has no control over thereby usage of this data for determining the state of health 5, 6 of an individual is much more accurate.

The data received by the various body sensors 2 is provided to the extraction unit 15 which processes the sensor data to generate body data 3. The body data 3 is provided to the primary processing unit 4 which compares the body data 3 to reference values 7 for determining the state of mental health 5 of the user can the stress levels 6 which the user is undergoing. It further generates a body score 8 using the body data 3. The said body score 8 pertains to mental health, physical health, heart health, sleep, activity of the human body and the overall health of an individual. The body score 8 indicate a person's actual state of mind and body at any given point, including while carrying out a focused activity like a psychologist-suggested therapeutic activity. It gives insights to the user regarding the actual time that user became relaxed as a result of a stress reduction activity. The body data 3, body score 8 and the data regarding the state of health 5, 6 is provided to an IOT unit 17 which further communicates the data to a remotely placed database 16 for retrieval at some future point of time. Alternatively, the data may be stored within the primary processing unit 4 or an internal storage which is separate from the primary processing unit 4. The data may also be stored in a removed drive such as a microSD card, flash drive etc. The data can be directly transmitted to an intermediate device via an appropriate data transmission system. This intermediate device may store the data or pass it further to a long term storage unit, which could be a virtual unit in the cloud or a physical unit. The data could be intermittently or periodically transmitted to a storage unit and stored based on automatic scheduling by the remote central monitoring unit. Both raw and processed data and information can be stored.

The environmental sensors 19 provided detect environmental information including the geographic location 22, altitude, weather 22, food and nutrition habits 24 and surroundings 21. The contextual data 20 generated by the sensors 19 is provided to the primary processing unit 4 which processes it along with the body data 3 to generate body score 8 and derive the state of health 5, 6 of the user. The contextual data 20 can be gathered on demand from any other authorized and external or third party IoT devices and applications. The body data 3 and contextual data 20 is provided to the Machine Learning and Artificial Intelligence unit 25 for analysis and derivation of patterns related to everyday activities, stressors and triggers in different states and environments. It further compares patterns 33 in different user states and environments. Additionally, it determines user's response to stimulus and therapies and generates risk stratification 26 based on users' response to different stimulus. The Machine Learning and Artificial Intelligence unit 25 makes dynamic usage of statistical methods and predictive algorithms for providing dynamic recommendation of different therapeutic 27 options based on user behaviour, response to stimulus, response to therapy, contextual information, it initiates action based on risk stratification 26 and perceived deterioration of physical and mental health of user. It also activates emergency protocol and interventions are recommended based on processed data. Based on the processed data, the Machine Learning and Artificial Intelligence unit 25 also provides feedback to the user, such feedback may be a reminder or an alert to eat a meal or take medication or a supplement such as a vitamin, to engage in an activity such as exercise or meditation, or to drink water when a state of dehydration is detected. Additionally, a reminder or alert can be issued in the event that a particular physiological parameter such as ovulation has been detected, a level of calories burned during a workout has been achieved or a high heart rate or respiration rate has been encountered. In an embodiment, the system is provided as a wearable device 28 which can be worn on the hand or feet of the user as per user's preference. The device 28 is incorporated with the sensors 3, 19, primary processing unit 4, IOT unit 17, input unit, output unit 31, the Machine Learning and Artificial Intelligence unit 25 along with battery 29 and a battery management unit 30. Alternatively, the primary processing unit 4 or IOT unit 17 or Machine Learning and Artificial unit 25 or all of them may reside outside the device. The wearable device 28 can be connected IoT devices, storage devices, third party systems, databases, cloud environment and so on via a transmission system. There exist several connectivity options for IoT devices, both wired and wireless. Depending on the usage, including application, range, data requirements, security, power demands and battery life any one or the some form of combination of technologies could be used to transmit the data. Options include, but not limited to BlueTooth, BlueTooth Low Energy (BLE). ZigBee. Z-Wave, 6LowPAN, Thread. Wifi, Cellular, Near Field Communication (NFC), Sigfox, Neul. LoRaWAN The primary processing unit may be placed within another IOT enabled device or distributed in a cloud environment, and can connect wirelessly to the wearable device by means of any electronic data communication protocol including, but not limited to to BlueTooth, BlueTooth Low Energy (BLE), ZigBee, Z-Wave, 6LowPAN, Thread, Wifi, Cellular, Near Field Communication (NFC), Sigfox, Neul, LoRaWAN. In another alternative, the primary processing unit is not present and its processing operation is performed by a mobile phone, a gateway device or any other internet-enabled devices owned by the owner of the wearable device. Alternately, the processing function can be performed collectively in a data center or within a large network Further, the primary processing unit 4 may be an independent unit, residing outside the wearable device 28 and communicating with the wearable, the cloud environment and other connected devices and environments on its own. The central monitoring will generate real-time analytics indicative of data from at least one of the sensors. The data will be accessible by the recipient over an electronic network. Real-time data transmission with integrated telemedicine platform.

The battery 29 provided in the wearable device 28 is a rechargeable battery which is managed by a battery management unit 30 that protects the battery 29 from operating outside its Safe Operating Area, monitors its state, calculates secondary data, reports the said data, controls the environment of the battery 29, authenticates and balances it. The battery management unit 30 maybe battery chargers, fuel gauges, battery monitors, battery selectors, and battery protectors which reduce cost, save space, and significantly extend the battery life. The battery management unit should provide safe charging for 1- to 4-cell Li-Ion (Li+)/Li-Polymer, NiMH/NiCd, lead-acid and rechargeable batteries of other chemistries in various sizes. The fuel gauges monitor remaining battery charge using algorithms like the proprietary ModelGauge™ algorithm to provide the highest accuracy. SHA-256 authentication helps to prevent battery pack cloning. The battery charger ICs provide support for USB Type-C devices, meeting all Power Delivery (PD) 3.0 voltage range specifications. Together, battery chargers and fuel gauges provide the efficiency, accuracy, and protection required to support Li+ battery applications. High-efficiency switching battery chargers provide low-heat and fast-charging solutions up to 9A for high-capacity batteries. Different charging options include: Battery charger ICs with integrated fuel gauge offer small solution size and simplify the system software design. USB Type-C chargers with integrated CC detection and BC1.2 detection offer a single-chip solution for USB Type-C and legacy USB systems. Different battery technologies include, not limited to Nickel Cadmium (NiCd) battery, the Nickel-Metal Hydride (NiMH) battery, Lead Acid battery, Lithium Ion battery, Lithium Polymer battery and so on.

The body data 3, body score 8, recommendations 27, 26, 33 and information on the state of health 5, 6 of the user are provided to the output unit 31 provided in the device 28 which is presented to the user as text, video or voice messages. The input unit provided is used for receiving data for validation of the output data generated. The input unit is equipped to recognise the voice of the user, detect signals as well as interpret them for automatic validation and determine the manual assessment data provided by the user for validation of the output data. The moods of the user, interests including combination of mental and physical activities, adherence to goals and recommended exercises, etc., responses to questions posed periodically also form part of the validation performed by the user. Alternatively, the output data may also be remotely accessible or the output unit 31 may reside outside the wearable device 28 such as smart watch, smart phone, smart kiosk, smart panels on cars, fridges, tabletops and electronic display boards. The output data can be downloaded, shared socially, stored, archived, interpreted or used as input to a separate system independent of the present invention.

In another embodiment, predictive algorithms based on streaming analytics are utilized for diagnosis, treatment and monitoring the state of health of a human body.

Further the device 28 is connected to a remote telemedicine platform 32 for facilitating users connection with caregivers, healthcare and emergency care professionals before, during and after any events. Through this platform 32 users can approach appropriate people, and also stream physiological data in real-time so that immediate inferences can be made by doctors or healthcare teams. Reports showing current and past status, trends, anomalies, history and so on can also be generated on-demand and in real-time. The device 28 can also be connected with multiple websites, apps, dashboards, third party hardware or software systems that are enabled by IOT. A dedicated smart app can be used by users to view their own data, reports, therapies, specific information and guidance targeted to them and generic content. The said app could be installed in any smart system, including but not limited to smart phones, watches, kiosks, displays, panels and so on. Users can also view their own data, reports, therapies, specific information and guidance targeted to them and generic content as web app or in browser in any internet enabled system with proper credentials Aggregate group information can be viewed. Body data which has been generated by the body sensors is also utilized to generate real-time analytics.

An authentication system is provided which pertains to making the hardware of the device tamper resistant, providing proactive and periodic firmware updates, performing dynamic testing to identify data tampering and suspected activities and specifying ways to protect data on device disposal.

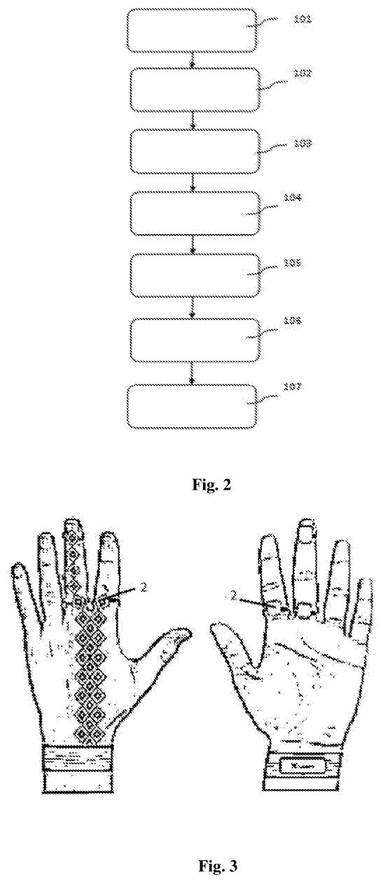

FIG. 2 shows a flow diagram for determining the mental health and stress levels of a user. In step 101, the physiological data is obtained by the body sensors to generate body data. In step 102, the body data is utilized to generating a body scores that pertains to the mental health, physical health, heart health, sleep health, activity score and overall score by comparing the body data with pre-defined reference values. Based on the algorithms, the mental health and/or stress levels of a user are determined. In step 103, the body data, body score, mental health and stress level data is provided to an IOT unit which is capable of storing the data on a remote database for further processing. In step 104, the contextual information including geographic location, food & nutrition habits, environment and interaction with other humans is received and processed by the environmental sensors to generate contextual data. In step 105, the body data and contextual data are analyzed to identify patterns of the human body and compare the identified pattern against reference values to determine the health risks that a user might potentially face.

In step 106, the health risk data is combined with the risk stratification of the user which is obtained based on response generated based on various stimulus and therapies administered on human body. The combined data is utilized for providing the user with therapeutic recommendation. In step 107, the therapeutic recommendation, body score as well as information on mental health and stress levels and feedback on the current state of health of a user is provided as output to the user.

FIG. 3 shows the plurality of wearable sensors 2 used by the system to obtain physiological data of a user's body. In an embodiment, the wearable device is designed as a glove which a user can wear on their hands. The wearable sensors 2 in the glove collects physiological data from a user while the person is relaxing and the data is analysed to determine the user's actual state of mind and body. The wearable sensors include Electrodermal activity sensors (EDA), PPG sensors ECG sensors, EMG sensors, skin temperature sensor, motion sensor as well as heat sensors. EDA can be placed at any two points near to the palm or fingers in the wearable, it can also be placed on feet to collect similar type of data. The accuracy of EDA being placed on hand and feet are almost similar. PPG can be placed near to any of the finger tips and on the wrist in the wearable. PPG can also be placed near to the ear lobe, toes and wrist. The accuracy of PPG being placed near to finger tips and ear lobe is almost similar. Any other site of placement reduced the accuracy of the reading. Accelerometer can be placed anywhere in the wearable. The placement depends on the kind of movement that has to be measures. In one of the embodiment, the movement of hand is required to be determined, so sensor is placed near to the dorsal side of the hand.

### Advantages of the Invention

The aforementioned invention is applicable in multitude of areas of the medical domain. Some of the areas where the invention can be effectively used include: Psychiatry—for assessment and intervention in anxiety disorders spectrum (GAD, phobias, panic, PTSD and OCD; childhood anxiety like test anxiety, separation anxiety etc) and Mood disorders (Depression, Mania, Bi-polar), identifying physiological and emotional arousal in children with neuro-developmental disorder (NDD), substance abuse and physiological markers in and children and adolescent, understanding physiological markers and therapeutic/rehabilitative strategies in geriatric care, including in dementia, Parkinson's & Alzheimer's. Dermatology-for self-monitoring, identifying trigger/stressor in dermatological diseases as well as psycho-dermatological disorders. Pain management—for pain assessment, behavioral assessment and interventions as well as assessment of pre-operative and post-operative patients. Cardiology—for non-cardiac chest pain: assessment and intervention, physiological and emotional arousal in various cardio-vascular disorders and psychological interventions. Neurology—for migraine, psycho-somatic tension headaches: Identifying and self-monitoring stressor/triggers and bio-feedback. Medicine—for self-monitoring (physiological and emotional arousal), assessment and intervention in medical conditions such as hypertension, diabetes, hyper/hypothyroidism etc. Oncology—for assessment of depression, health anxiety, death anxiety and interventions. Sports medicine—for physiological and emotional arousal before and after performance and interventions based on performance anxiety and related stressors. Military medicine—for identifying various stressors in new recruits during training and war like situations and development of relevant intervention modules, physiological and emotional arousal responses (self-monitoring) in PTSD and intervention for veterans and on duty soldiers. Healthy population—for stress monitoring in healthy population and relaxation techniques. Effectiveness of therapies and interventions—for determining the physiological and emotional responses of the individuals to various therapies.

While specific language has been used to describe the invention, any limitations arising on account of the same are not intended. As would be apparent to a person skilled in the art, various working modifications may be made to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

### LIST OF REFERENCE NUMERALS

1—System for management of a stress level and mental health of a human body
2—Body sensors
3—Body data
4—Primary processing unit
5—Metal health
6—Stress level
7—Reference values
8—Body score
9—Electrodermal activity sensor (EDA)
10—Skin temperature sensor
11—Photoelectric plethysmography sensor (PPG)
12—Electro cardiogram sensor (ECG)
13—Electro myography sensor (EMG)
14—9-axis motion sensor
15—Data extraction unit
16—Database
17—IoT unit
19—Environmental sensors
20—Contextual data
21—Interaction of the human body with other human bodies
22—Geographic location visited
23—Environment
24—Food and nutrition habits
25—Machine Learning and Artificial Intelligence processing unit
26—Risk stratification
27—Therapeutic recommendation
28—Device
29—Rechargeable battery
30—Battery management unit
31—Output unit
32 Telemedicine platform
33—Patterns identified by Machine Learning and Artificial Intelligence processing unit

1. A system (1) for management of a stress level and mental health of a human body, the system (1), comprising:
one or more body sensors (2) adapted to measure at least one of a physiological parameter of the human body, body movement of the human body, or heat expenditure of the human body or combination thereof, and to generate a body data (3);
a primary processing unit (4) adapted to receive and process the body data (3) and adapted to determine at least one of the mental health (5) of the human body and the stress level (6) of the human body.

2. The system (1) according to the claim 1, wherein the primary processing unit (4) is adapted to process the body data (3) by comparing the body data (3) with one or more reference values (7), and adapted to determine at least one of the mental health (5) of the human body and the stress level (6) of the human body

3. The system (1) according to the claim 1, wherein the primary processing unit (4) is adapted to process the body data (3) and to generate a body score (8) related to at least one of overall health, mental health, physical health, heart health, sleep, or human body activity, or combination thereof.

4. The system (1) according to the claim 1, wherein one or more physiological parameter of the human body is measured using at least one of electrodermal activity sensor (9), skin temperature sensor (10), photoelectric plethysmography sensor (11), electro cardiogram sensor (12), or electro myography sensor (13), or combination thereof.

5. The system (1) according to the claim 1, wherein the body movement of the human body is measured by a 9-axis motion sensor (14) which comprises a 3-axis gyroscope, 3-axis accelerometer and 3-axis magnetometer.

6. The system (1) according to the claim 1 comprising a data extraction unit (15) functionally coupled to the one or more body sensors (2), and to extract the body data (3) from the body sensors (2).

7. The system (1) according to the claim 1 comprising a database (16) adapted to receive and store at least one of the body data (3), the body score (8), or the determination of at least one of the mental health (5) of the human body and the stress level (6) of the human body.

8. The system (1) according to the claim 7 comprising an IoT unit (17) adapted to establish communication to the database (16), which is placed remotely or within the IoT unit (17), and adapted to send at least one of the body data (3), the body score (8), or the determination of at least one of the mental health (5) of the human body and the stress level (6) of the human body to the database (16) in periodic batches or real-time streams of data.

9. The system (1) according to the claim 1 comprising an input unit (18) adapted to receive inputs related to validation of at least one of the body data (3), the body score (8), or the determination of at least one of the mental health (5) of the human body and the stress level (6) of the human body;

10. The system (1) according to the claim 1 comprising one or more environmental sensors (19) adapted to generate a contextual data (20) related to at least one of interactions of the human body with other human bodies (21), geographic location visited (22) by the human body, environment (23) of the human body, or food and nutrition habits (24) of the human body, or combination thereof,
wherein the primary processing unit (4) is adapted to process the body data (3) along with the contextual data (20) for at least one of the determination of at least one of the mental health (5) of the human body and the stress level (6) of the human body, and generation of the body score (8).

11. The system (1) according to the claim 1 comprising a Machine Learning and Artificial Intelligence processing unit (25) adapted to receive and process the body data (3) and the contextual data (20), adapted to identify one or more patterns (33) of the human body based on processing of the body data (3) and the contextual data (20) and adapted to compare the patterns of the human body, and to determine risks related to stress level (6) and mental health (5) of a human body.

12. The system (1) according to the claim 1, wherein the Machine Learning and Artificial Intelligence processing unit (25) is adapted to receive various stimulus and/or therapies administered to the human body, and to generate responses to each of the stimulus and/or therapies by the human body based on the body data (3).

13. The system (1) according to the claim 12, wherein the Machine Learning and Artificial Intelligence processing unit (25) is adapted to process the responses to each of the stimulus and/or therapies by the human body and adapted to generate risk stratification (26) of the human body.

14. The system according to the claim 12, wherein the Machine Learning and Artificial Intelligence processing unit (25) is adapted to generate therapeutic recommendation (27) based on at least processing of the responses to the stimulus and/or therapies by the human body based on the body data (3), body score (8), or the contextual information (20), or combination thereof.

15. The system (1) according to the claim 1 comprising a device (28) which encapsulate at least one of the sensor/s (3, 19), the primary processing unit (4), the data extraction unit (15), the database (16), IoT unit (17), the input unit, or, the Machine Learning and Artificial Intelligence processing unit (25), or the combination thereof, wherein the device (28) is wearable on at least one of the body parts.

16. The system (1) according to the claim 15, wherein the device is wearable on hand or feet of the human body.

17. The system (1) according to the claim 15, wherein the device (28) further comprises a rechargeable battery (29) and a battery management unit (30), the battery management unit (30) is adapted to at least monitor a physical state and/or chemical state of the battery (29), to control operating environment of the battery (29), or combination thereof.

18. The system (1) according to the claim 15, wherein the device (28) further comprises an output unit (31) adapted to render at least one of the body data (3), the body score (8), or the determination of at least one of the mental health (5) of the human body and the stress level (6) of the human body.

19. The system (1) according to the claim 15 wherein the device (28) processing unit is adapted to be remotely coupled for communication to a telemedicine platform (32) for enabling data flow between the device and the telemedicine platform (31).

20. The system (1) according to the claim 1, wherein the primary processing unit (4) of the device (28) unit is adapted to measure the effectiveness of different types of mental health therapies in a quantitative way, including measuring the effectiveness of meditation and mindfulness along with other Cognitive Behavior Therapy.
